(19) United States
(12) Patent Application Publication
(10) Pub. No.: US 2024/0358319 A1
(43) Pub. Date: Oct. 31, 2024

(54) LOOP GAIN DETECTION AND APPLICATION TO BREATH TRAINING

(71) Applicant: Halare, Inc., Sewickley, PA (US)

(72) Inventors: Anthony C. Warren, Wien (AT); Matthew Michaux, Sewickley, PA (US)

(73) Assignee: Halare, Inc., Sewickley, PA (US)

(21) Appl. No.: 18/640,507

(22) Filed: Apr. 19, 2024

Related U.S. Application Data
(60) Provisional application No. 63/461,743, filed on Apr. 25, 2023.

(51) Int. Cl. A61B 5/00; A61B 5/024; A61B 5/145; G16H 20/30

(52) U.S. Cl. CPC A61B 5/4836; A61B 5/024; A61B 5/14542; A61B 5/681; A61B 5/7264; A61B 5/74; G16H 20/30; A61B 2560/0468

(57) ABSTRACT

A portable smart device, comprising a sensor coupled to the device, the sensor configured to sense a physiological parameter of a user, and a processor configured to perform a breath training session to determine breathing loop gain in an awake state by instructing the user to breathe in a specified manner, receive physiological data from the sensor, the physiological data representing the sensed physiological parameter of the user, and determine a value of the loop gain based on the physiological data from the sensor.

### TECHNICAL FIELD

The present disclosure relates to systems, methods and apparatuses for detecting breathing instabilities and using the results therefrom to aid trainees to alleviate symptoms of sleep disordered breathing, (SDB) including but not limited to sleep apnea and hypopnea.

### BACKGROUND

A proportion of Sleep Disordered Breathing (SDB) relates to a condition characterized by repeated episodes of hypopnea (under-breathing) and apnea (not breathing) during sleep resulting in reduction in blood oxygen saturation (SpO2), arousal from sleep and sympathetic nervous system activation. Pauses may last 10-20 seconds or more and can occur 20 to 30 times or more an hour. A common type of SDB affecting approximately 85% of trainees is referred to as Obstructive Sleep Apnea (OSA) in which physical obstruction of the airways occurs due to sleep related loss of upper airway dilator muscle tone. A second type of SDB, commonly referred to as Central Nervous System Apnea (CNSA) is less common and occurs when the brain region that controls breathing fails to send signals to breathing muscles in a timely manner. It is now generally accepted that CNSA often occurs in combination with OSA and that the historical separation of OSA and CNSA insufficiently categorizes the disease forms and therapies.

OSA events may be triggered by one or more of at least four different causes as shown in FIG. 1. In many cases, mechanical restriction of the upper airways is present, particularly in obese individuals 1.2. This may be exacerbated by weak upper airway musculature 1.4. leading to airway collapse while asleep. A third cause, 1.6, is a propensity to awake easily, referred to as having a ‘low arousal threshold’. In this case, small disturbances including breathing irregularities may cause an apnea event. A fourth cause is referred to as high loop gain (LG) in the breathing control system, 1.8. Triggers 1.4, 1.6 and 1.8, each have a probability of about 35% or greater of being present in a sufferer of OSA during an OSA episode.

In general, SDB can have short-term and long-term deleterious impacts. When sleep is interrupted throughout the night, drowsiness occurs during the day. People with SDB have twice the risk for car accidents, are 25% more likely to have at-work accidents and exhibit loss of work efficiency. SDB may also lead to long-term serious, chronic health issues, such as increased chance of stroke and other cardio-vascular diseases, and dementia. Roughly 38,000 cardiovascular deaths annually are in some way related to SDB. SDB may be undiagnosed in many cases until symptoms have become life threatening. Estimates are between twelve and twenty million Americans suffer from SDB. The economic impact of SDB in the United States alone is estimated to be several billion dollars annually, which does not take into account the cost of long-term care associated with other chronic diseases triggered by SDB.

### SUMMARY

In an example, the disclosure includes a portable smart device, comprising a sensor coupled to the device, the sensor configured to sense a physiological parameter of a user, and a processor configured to perform a breath training session to determine breathing loop gain in an awake state by instructing the user to breathe in a specified manner, receive physiological data from the sensor, the physiological data representing the sensed physiological parameter of the user, and determine a value of the loop gain based on the physiological data from the sensor.

In an example of the disclosure, the sensor is at least one of a heart rate sensor or a blood oxygen saturation sensor.

In an example of the disclosure, the processor is further configured to guide the breath training session by instructing the user to breathe in a specified manner by performing breath holds and rhythmic breathing patterns intended to reduce the loop gain.

In an example of the disclosure, the processor is further configured to track changes in the loop gain over time.

In an example of the disclosure, the processor is further configured to determine a value of the loop gain based on a phenotype classification of the user.

In an example of the disclosure, the sensor is integrated directly into the portable smart device.

In an example of the disclosure, the sensor is external to and in communication with the portable smart device.

In an example of the disclosure, the processor is further configured to instruct the user to perform breathing exercises for the determination of the loop gain.

In an example of the disclosure, the processor is further configured to classify the user as having a high loop gain based on the determined value of the loop gain and previously determined physiological data.

In an example of the disclosure, the processor is further configured to tailor breath training exercises specifically to reduce the high loop gain, the tailoring based on the classification of the user and the determined value of the loop gain.

In an example, the disclosure includes a method for determining breathing loop gain in an awake state using a portable smart device, the method comprising sensing a physiological parameter of a user with a sensor coupled to the device, performing a breath training session to instruct the user to breathe in a specified manner, receiving physiological data from the sensor, the physiological data representing the sensed physiological parameter of the user, and determining a value of the loop gain based on the physiological data from the sensor.

In an example of the disclosure, the sensing step involves using at least one of a heart rate sensor or a blood oxygen saturation sensor.

In an example of the disclosure, the method further comprising guiding the breath training session by instructing the user to breathe in a specified manner by performing breath holds and rhythmic breathing patterns intended to reduce the loop gain.

In an example of the disclosure, the method further comprising tracking changes in the loop gain over time.

In an example of the disclosure, the method further comprising determining a value of the loop gain based on a phenotype classification of the user.

In an example of the disclosure, the sensor is integrated directly into the portable smart device.

In an example of the disclosure, the sensor is external to and in communication with the portable smart device.

In an example of the disclosure, the method further comprising instructing the user to perform breathing exercises for the determination of the loop gain.

In an example of the disclosure, the method further comprising classifying the user as having a high loop gain based on the determined value of loop gain and previously determined physiological data.

In an example of the disclosure, the method further comprising tailoring breath training exercises specifically to reduce the high loop gain, the tailoring based on the classification of the user and the determined value of loop gain.

### BRIEF DESCRIPTION OF DRAWINGS

FIG. 1 depicts four triggers that may initiate sleep disturbed breathing events, according to aspects of the disclosure.

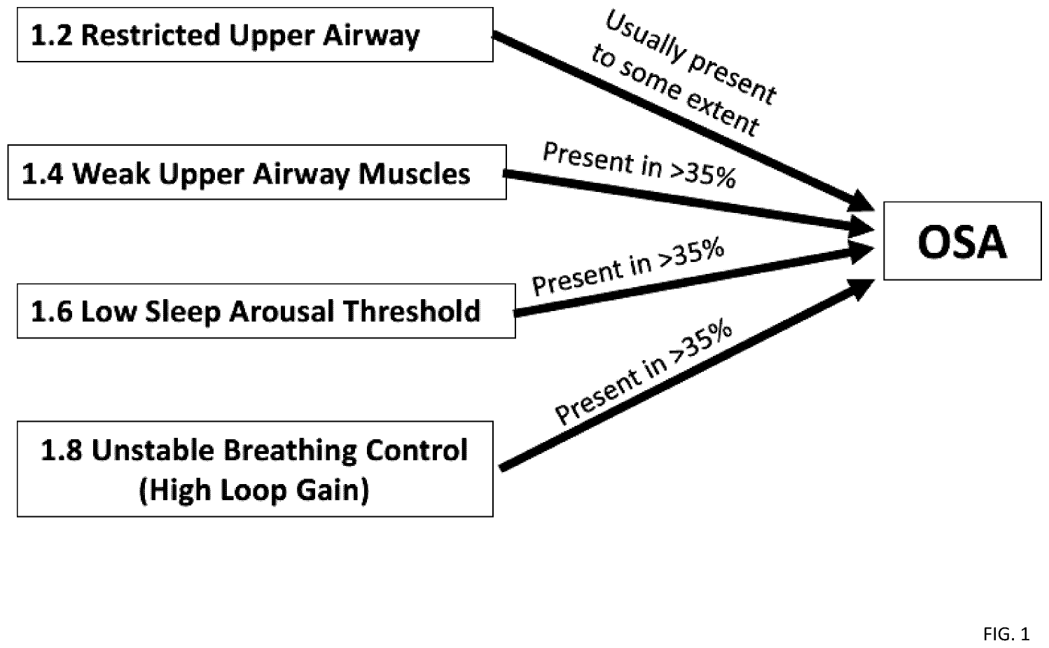

FIG. 2 is a schematic diagram of the breathing control system showing how the controller components are linked in a feedback loop with an inbuilt delay, according to aspects of the disclosure.

FIG. 3 depicts an example of how the system of FIG. 2 may exhibit instability when the loop gain is above 1, according to aspects of the disclosure.

FIG. 4 depicts several formats of sensing systems used for home sleep apnea tests, according to aspects of the disclosure.

FIG. 5 depicts several different sensing devices that can be used to determine loop gain, according to aspects of the disclosure.

FIG. 6 depicts an example of a CPAP Drop Sequence, according to aspects of the disclosure.

FIG. 7 depicts a time chart of inhale and exhale airflow cycles interspersed with a 20 second breath holding interval immediately followed by cycles of unstable and increased breath cycles, according to aspects of the disclosure.

FIG. 8 depicts time charts of airflow and oxygen saturation values interspersed with an extended forced breath hold interval, according to aspects of the disclosure.

FIG. 9 depicts how pulse rate data is used to determine breath hold times, according to aspects of the disclosure.

FIG. 10 depicts the relationship between LG and post-breath-hold Airflow for three phenotypes, according to aspects of the disclosure.

FIG. 11 depicts the relationship between LG and breath-hold time for three phenotypes, according to aspects of the disclosure.

FIG. 12 depicts an example of loop gain decline from breath training over several weeks, according to aspects of the disclosure.

### DETAILED DESCRIPTION

Loop gain (LG) is a term used to measure the stability of the negative feedback chemoreflex breathing (or ventilatory) control system. The overall LG of this system reflects the ratio of the ventilatory response to the disturbance that triggered the response. When breathing deviates from a balanced state (i.e., in which breathing activity matches metabolic demand), such as during a hypopnea, if the ventilatory response that is elicited by the disturbance does not exacerbate the deviation, (LG=≤1) ventilation will correct blood gases to re-establish steady state balanced levels. If the ventilatory response is disproportionately larger than the disturbance (LG>1), ventilation will not only correct the disturbance to blood gases but will overshoot such that the partial pressure of carbon dioxide in arterial blood, (PaCO2) which will be reduced below normal levels. This results in hypoventilation, upper airway muscle slackness and a secondary airway obstruction such that respiratory events become self-perpetuating in a vicious cycle. Higher LG reflects less stable ventilatory chemoreflex control.

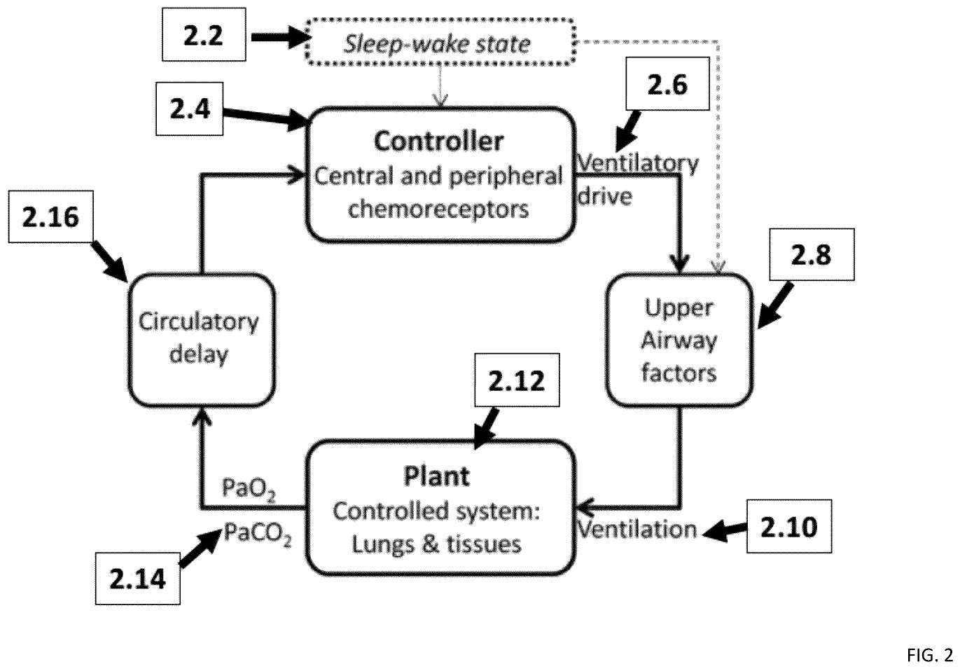

Control loop theory dictates there are both a controller and a plant component, with a delay time between the two. FIG. 2 is a schematic of the breathing control system. The system works to maintain a balance between breathing performance and metabolic needs in both sleep and awake modes 2.2. A controller 2.4 senses blood gas concentrations, CO2 and O2 from a number of centrally and peripherally located chemo-sensors and determines appropriate stimulus signals 2.6 and 2.10, taking into account prevailing upper airway factors, 2.8. The breathing muscles are stimulated by the plant 2.12, to return CO2 and O2 in the blood to normal balanced values 2.14. There is an inherent delay 2.16 before the system can reach equilibrium. In ventilatory control, chemoreceptor sensitivity to blood gases reflects controller gain, and the effectiveness of the lungs to alter blood gases reflects plant gain. The product of controller gain (CG) and plant gain (PG) provides the overall LG of the system (LG=CG×PG). Because there is a circulation delay between when ventilation begins to modify blood gases and when the chemoreceptors sense the change, if the gain of either the controller or the plant is too high, there is the potential for ventilatory overshoot producing instability in the system.

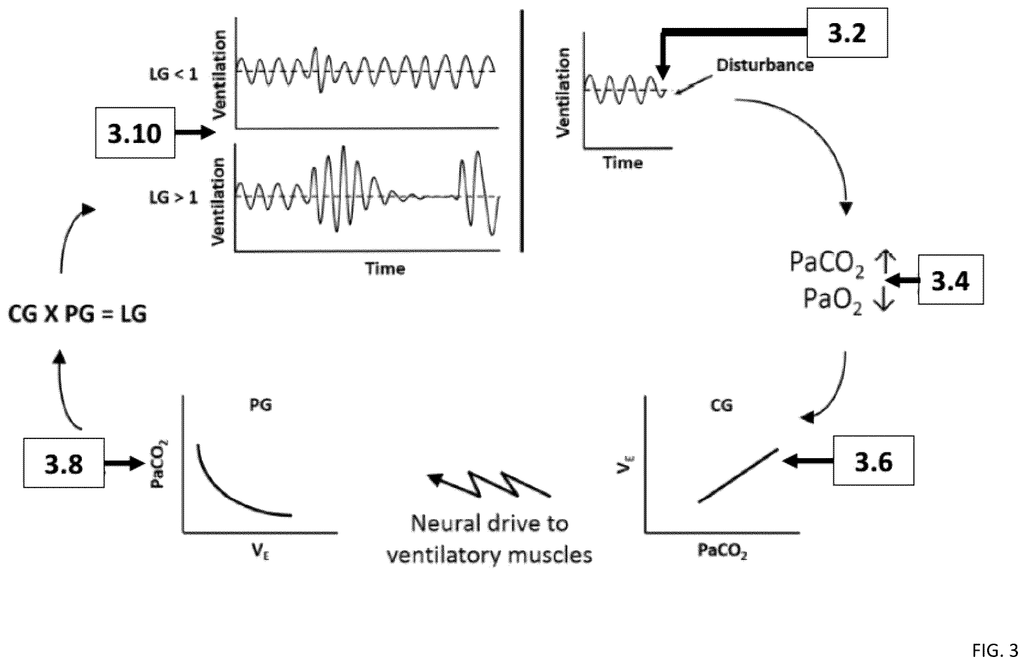

FIG. 3 is a schematic of how LG impacts breathing stability. At 3.2 a breathing disturbance triggers a reduction in air flow below the body's needs. At 3.4 such a reduction eventually increases the level of carbon dioxide in the blood (PaCO2) concurrently reducing the oxygen level, O2 (PaO2). At 3.6 chemical sensors detect these changes in blood chemistry and the controller Gain (CG) dictates the magnitude of neural drive to the breathing muscles. At 3.8 the plant gain (PG) represents the effectiveness of the lungs to change blood gases, CO2 and O2. At 3.10, the product of CG and PG determines overall LG. If LG<1, the breathing fluctuations will dampen out and stabilize. If LG>1, the fluctuations will increase in amplitude and instability will be self-perpetuating resulting in the repetition of apneic or hypopneic events experienced in Obstructive Sleep Apnea (OSA) or disturbed breathing in the wakened state.

In order to determine whether high LG is a possible contributive cause of sleep disordered breathing (SDB), a means to measure the LG value inexpensively and simply would be beneficial, preferably at a place of choice of the sufferer of OSA. Furthermore, a known value of LG determines whether a therapy to reduce a high LG is appropriate, and if so, to define the parameters of such therapy. The disclosure herein describes such a method for the determination of LG 1.8, for a person while awake at place of their choice and applying the measurements to personalize the alleviation of OSA to improve therapeutic outcomes. Currently, determination of LG during diagnoses of the causative factor of OSA is rarely undertaken because it requires a complex and costly procedure. Thus, current means are not available to tailor OSA therapy to match the underlying cause arising from elevated LG. This, in turn, results in reduced positive therapeutic outcomes, which can be life-threatening.

Breathing control in humans is a complex system and is controlled from different locations in the brain. It is possible to modify breathing patterns when conscious, for example by voluntarily stopping breathing, breathing more slowly, deeper etc. These volitional actions are controlled within the motor cortex which resides at the top of the brain. However, when asleep, or not voluntarily modifying breathing patterns, autonomous breathing control takes over; humans continue to breathe naturally to maintain homeostasis while asleep. Autonomous breathing is controlled primarily from the medulla oblongata, a primitive part of the brain located at the brain stem. There is evidence that these two control centers can interact. For example, it has been shown that prompting small gradual changes in breathing rate are retained when the prompts are removed. Volitional modifications can alter subsequent autonomous control.

Imposing carefully designed hypoxia sequences may invoke neuroplastic changes in the breathing control centers in the brain that can mitigate the periodic apneas and hypopneas that occur in OSA and CNSA by reducing a high value of LG in order to stabilize breathing patterns during sleep. Descriptions of such breathing exercises that can be used to reduce LG are disclosed in U.S. Pat. No. 9,830,832 which is incorporated by reference herein for all purposes.

The disclosure herein provides a simple, inexpensive means to determine LG. The data that is collected from such determinations enables the selection of sufferers from a larger population likely to benefit from neuroplastic modifications of the breathing control centers. This avoids unnecessary therapeutic procedures. Moreover, the derived data from determining LG enable the breathing exercise regimens to be personalized for an individual, thereby improving the effectiveness and efficiency of treatment for those sufferers likely to benefit from breath training for the alleviation of SDB.

This disclosure relates to systems, methods, and apparatuses for aiding sufferers from SDB including sleep apnea and hypopnea, determining whether LG is a contributing factor to the SDB and, if so, guiding the sufferers through breath training regimens (e.g. exercises including breath holds, rhythmic breathing patterns, etc.) intended to reduce or eliminate symptoms of SDB resulting from an elevated value of LG.

The approach detailed herein leverages the accessibility and convenience of portable smart devices and/or other common sensors to facilitate the identification and management of SDB. By utilizing readily available sensors to monitor physiological parameters, individuals can engage in breath training sessions in the comfort of their own environment, without the burden of specialized medical equipment or the presence of healthcare professionals. This democratization of SDB management empowers individuals to take proactive steps in monitoring their condition, potentially leading to early intervention and improved health outcomes. The personalized nature of the breath training regimens, informed by the user's specific loop gain data, ensures that each individual receives a tailored therapeutic experience, optimizing the potential for successful treatment and symptom alleviation.

One technique to determine the LG for a control system is by introducing a perturbation into the system while the system is performing in a steady state. One method used for determining breathing related LG, employs a continuous positive airway pressure (CPAP) device manipulated during sleep in a procedure referred to as “CPAP drop.” This has a number of disadvantages as described below.

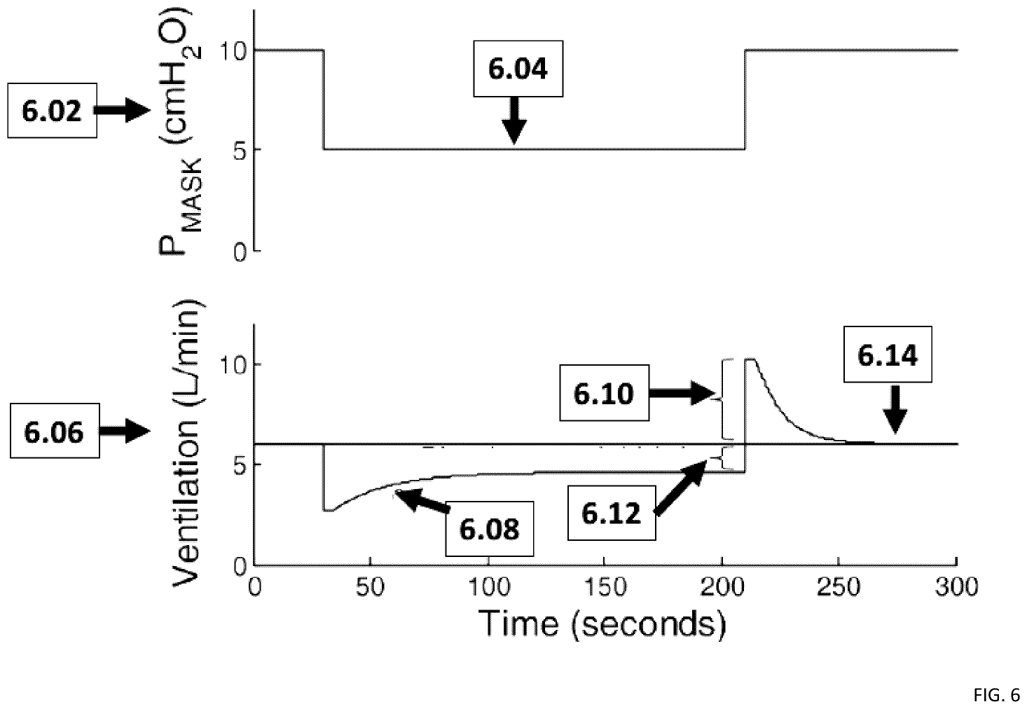

In one example of a CPAP drop, the sufferer is fitted with a CPAP mask, 504. During sleep, the air-flow sensors within the mask determine when the airway is fully open and breathing is regular and meets the normal metabolic demand. The pressure applied by the CPAP is then reduced or ‘dropped’, whereupon the upper airway narrows and air intake drops significantly or even ceases completely. After some time, the decreased ventilation leads to an increase in carbon dioxide leading to enhanced ventilatory drive that stimulates the diaphragm and the pharyngeal muscles. In some sufferers this causes air intake to recover but not completely. After a defined time period the CPAP is returned to the earlier pressure, the airway opens and the increased ventilatory drive produces an overshoot in airflow. The magnitude of such overshoot depends directly on the value of LG at that time. Eventually, ventilation and ventilatory drive return to a normal relaxed status because the airway is now fully open and breathing can once again match the metabolic demand. LG is calculated by dividing the ventilatory overshoot by the steady state reduction in ventilation. FIG. 6 shows one specific example of a CPAP drop sequence. 6.02 is a time chart of the pressure in centimeters of water applied by the CPAP mask. 6.06 is the concurrent time chart of the airflow passing through the upper airway in liters per minute. At time 30 seconds, the applied pressure is halved from 10 to 5 cm. of water and sustained at this value for 3 minutes, 6.04. The airflow at 6.08, decreases from 6 to 2.7 liters/min immediately at the drop, recovers somewhat at 6.12, to 6.6 liters/min in this example and then overshoots beyond normal demand to 10.2 liters/min at 6.12, before gradually returning to a balanced regular airflow at 6.14. LG is calculated by dividing the ventilatory overshoot of 4.2 liters/min by the steady state reduction in ventilation of 1.4 liters/min. In this example therefore, LG=3. At this value, LG is likely to be a trigger for OSA and it is therefore appropriate to use therapies to reduce LG. One such therapy is the application of breath training as described in U.S. Pat. No. 9,830,832.

The CPAP drop method requires a sufferer to be fitted with a CPAP mask, and have the said mask programmed for both therapeutic and LG determination purposes. Thus, the expense of obtaining, fitting and programming a CPAP device is expended before determination whether therapy is even required and, if so, which therapies are appropriate. Additionally, the sufferer should obtain a good fit to the CPAP mask and be comfortable in wearing the device during sleep. Unfortunately, many sufferers experience difficulties in adapting to a mask and compliance in usage is low. Therefore, a means for determining the value of LG prior to determining appropriate therapeutic regimens, and before prescribing and fitting a CPAP device would be beneficial.

In other words, the CPAP drop technique, while effective in measuring LG, requires the sufferer to be asleep, which usually requires overnight monitoring in a controlled environment such as a sleep laboratory or at home with specialized equipment. This can be inconvenient and uncomfortable for the sufferer, potentially affecting the quality of sleep and the accuracy of the measurement. Additionally, the technique's reliance on a CPAP device means it is not suitable for individuals who have not been prescribed CPAP therapy or who are intolerant to CPAP usage. Therefore, alternative methods for determining LG that can be performed in an awake state and without the use of a CPAP device are desirable to broaden the accessibility of this diagnostic measure.

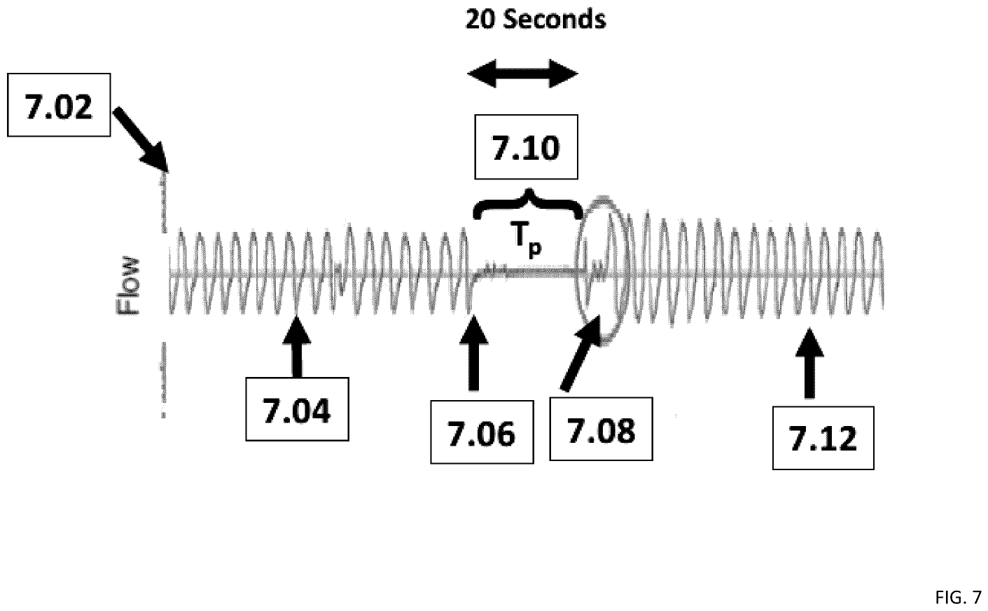

A first alternative method for creating a perturbation in the breathing control loop is for a sufferer to voluntary stop breathing for a pre-determined time, Tp, while in the awake state. FIG. 7 shows how airway flowrate 7.02 varies when breath-holding occurs. 7.04 shows regular inhale and exhale cycles prior to the perturbation followed by a prompted 20 second breath-hold Tp 7.10 commencing at 7.06, return to breathing occurs after 20 seconds at 7.08 whereupon the sufferer reverts to normal balanced breathing at 7.12. The transition from 7.08 to 7.12 provides beneficial information about the instability and hence the LG of the breathing control system. Specifically, the flow rate in the airway at the second inhale, FR2 as compared with a normal rest breathing rate value FRn, is related directly to the LG. The relationship between the ratio of these two values, FR2/FRn, expressed as a percentage and LG is defined in a set of look-up tables determined empirically from measurements on a statistically relevant population of at least N (e.g. 10) phenotype classes.

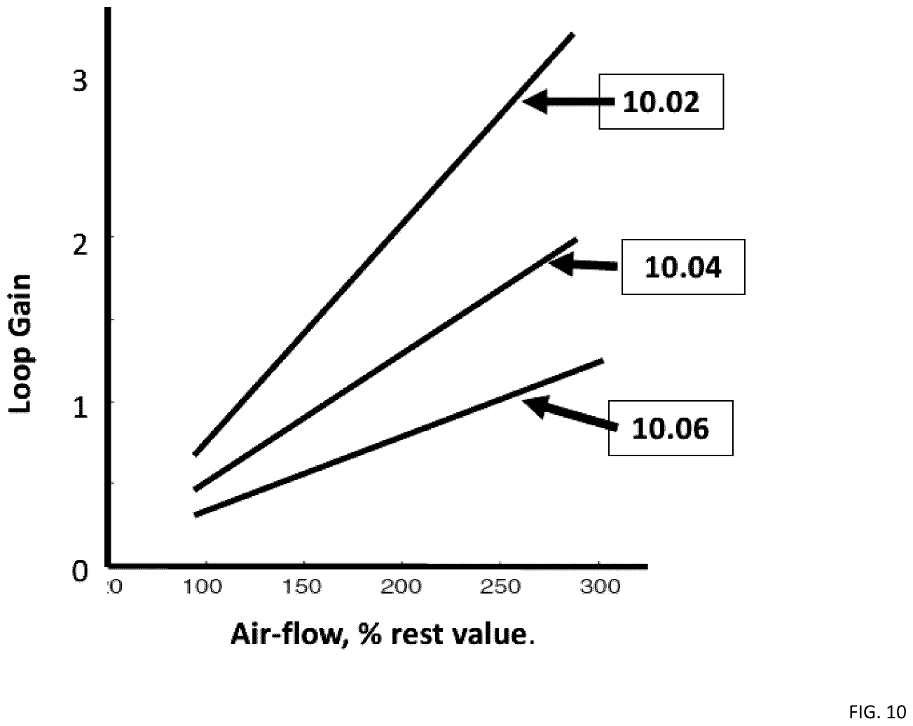

Each table in the set relates to a phenotype of one of these classes. Phenotype matching (i.e. classification) is based on individual personal and physiological data known to be correlated with SDB including but not limited to body mass index (BMI), age, gender, exercise activity level, smoking level, asthma intensity etc. An example of such a table represented in graphical form is shown in FIG. 10. The relationship between LG and FR2/FRn for three different phenotype categories, 10.02, 10.04, and 10.06 is shown. Once a sufferer has been assigned to a phenotype category and FR2/FRn determined by the methods disclosed herein, LG can be determined from such charts.

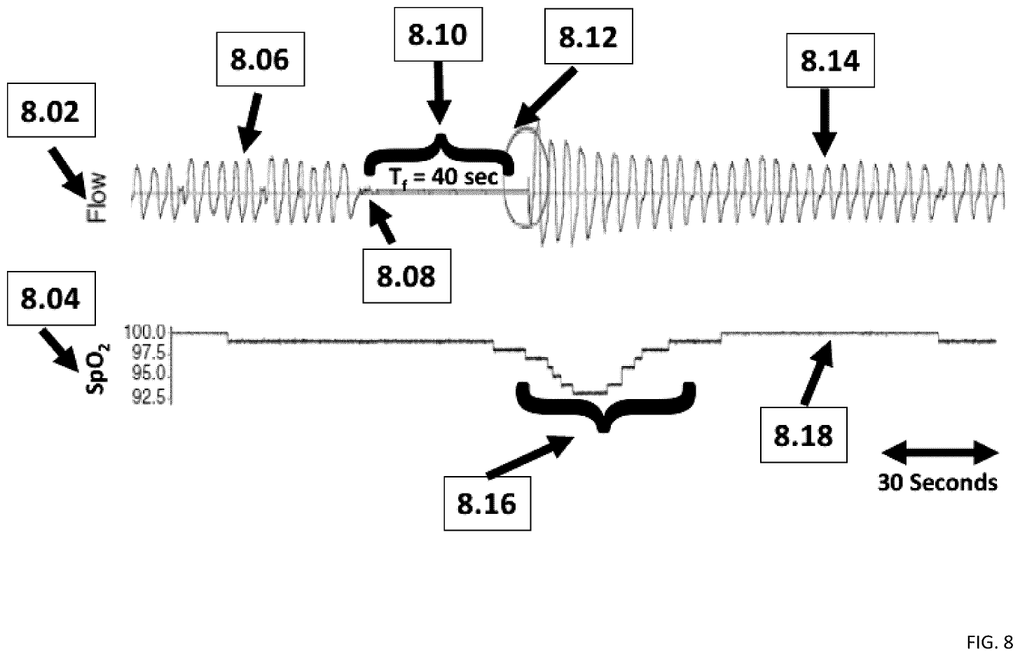

A second alternative method for creating a perturbation in the breathing control loop is for a sufferer to voluntary stop breathing while in the awake state. FIG. 8 shows how airway flowrate 8.02 varies when breath-holding occurs in this example. 8.04 shows regular inhale and exhale cycles prior to the perturbation followed by an extended, forced breath-hold Tf starting at 8.08. In this case, the breath-hold, 8.10, lasts 40 seconds commencing at 8.08 and ending at 8.12. The sufferer reverts to normal balanced breathing at 8.14. The transition from 8.12 to 8.14 provides beneficial information about the instability and hence the LG of the breathing control system. Specifically, the flow rate in the airway at the second inhale, FR2 as compared with a normal rest breathing rate value FRn, is related directly to the LG. The relationship between the ratio of these two values FR2/FRn, expressed as a percentage and LG is defined in a set of look-up tables determined empirically from measurements on a statistically relevant population of at least N (e.g. 10) phenotype classes. Each table in the set relates to phenotype one of these classes. Phenotype matching is based on individual personal and physiological data known to be correlated with SDB including but not limited to body mass index (BMI), age, gender, exercise activity level, smoking level, asthma intensity etc. An example of such a table represented in graphical form is shown in FIG. 10. The relationship between LG and FR2/FRn for three different phenotype categories 10.02, 10.04, and 10.06 is shown. Once a sufferer has been assigned to a phenotype category and FR2/FRn determined by the methods disclosed herein, LG can be determined from such charts.

In other words, similar to the first alternative method, the second alternative method, as shown in FIG. 8, also employs a voluntary breath-hold while awake but extends the duration of the breath-hold (Tf). This method similarly analyzes the airway flow rate changes post-breath-hold to infer the LG. However, it differs in that it uses an extended breath-hold to potentially elicit a more pronounced response in the breathing control system, which may provide a more distinct measure of LG. The resulting data is again matched to phenotype-specific look-up tables to determine LG. Both methods offer a non-invasive and awake-state alternative to the CPAP drop technique, enabling LG determination without the use of a CPAP device and the associated challenges of sleep-based measurements.

One example of instructions for a sufferer to execute a sequence of forced breath holds is: Step 1: Breathe normally for 3 minutes. Try to keep the mouth closed; Step 2: Breathe normally, then when prompted hold the breath after exhaling until the first clear urge to breathe is felt. Then take the first breath in through the nose calmly. Try not to gasp. (This is a self-imposed perturbation event); Step 3: Breathe normally for approximately five minutes, until prompted to hold the breath after breathing out. Hold the breath until the first clear urge to breathe is felt. Then take the first breath through the nose calmly. Try not to gasp. (This is the second self-imposed perturbation event); Step 4: From normal breathing, when prompted, gradually slow down until breathing is as slow as comfortable. Aim for about 6 breaths per minute. Count in for 4 seconds and out for 6 seconds; Step 5: From slow breathing, exhale, hold the breath until a strong need to breathe is felt. (This is the third self-imposed perturbation event); Step 6: Repeat steps 3,4,5 in sequence 2 or 3 times; and Step 7: Breathe normally for 3 minutes to relax. Keep the mouth closed.

When the second alternative method is employed using a forced breath-hold Tf, the value of Tf may be used to determine LG. In this case Tf must be independently determined. One means is to use a timer operated by the sufferer. However, activation of a timer can influence concentration on following the breathing instructions and it is therefore preferable to use one or more sensors to detect physiological parameters allowing the sufferer to focus on breathing regimen execution.

One such sensed physiological parameter is the concentration or oxygen in the blood, SpO2. When a forced breath-hold is executed SpO2 usually declines from a normal value of say 98% to as low as 85%. FIG. 8 illustrates the changes in this parameter 8.04 with a decline and recovery at 8.16. SpO2 declines from 98.5% to 92.5% and then recovers to a normal value at 8.18. The time interval, 8.16, closely maps to Tp although delayed. Thus, Tp may be determined using an SpO2 sensor with a sampling rate of, for example, at least 1 time a second and preferably greater than 5 times a second with a relative accuracy of +/−2%.

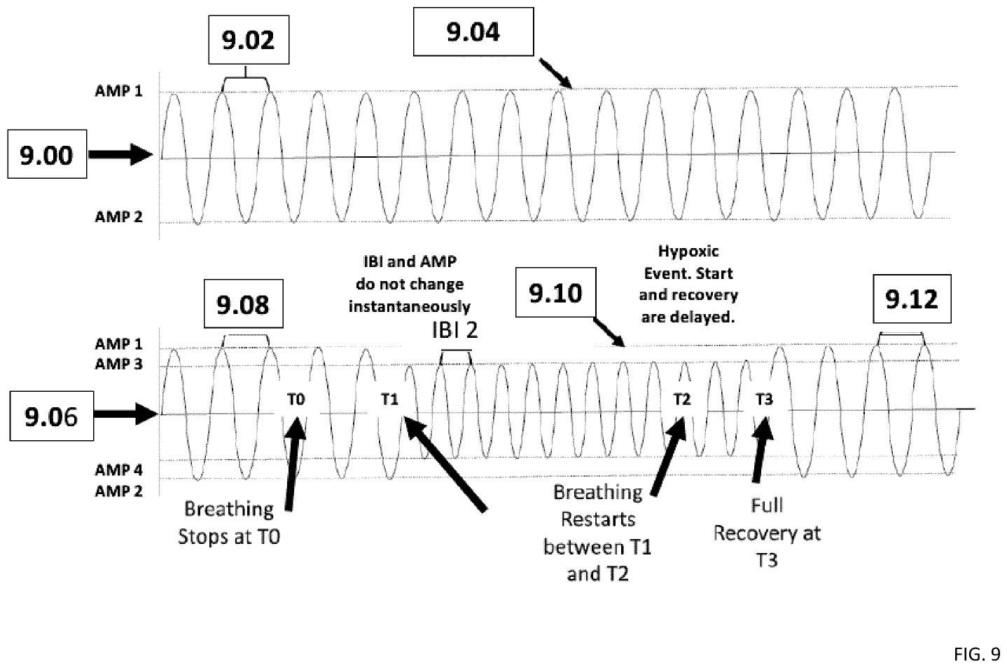

Another such sensed physiological parameter is the pulse rate as illustrated in FIG. 9. This is sensed using a simple optical technique to detect volumetric changes in blood in peripheral circulation known as Photoplethysmography (PPG). It is a low cost and non-invasive method that makes measurements at the surface of the skin. 9.00 is a schematic of a normal pulse trace cycling between two values, AMP 1 and AMP 2. It shows a regular interbeat interval, (IBI), 9.02 continuing at 9.04. When a forced breath-hold is executed the pulse trace is modified, 9.06. The sufferer first establishes a regular breathing pattern with IBI 9.08 and pulse magnitude defined by AMP 1 and AMP 2. The sufferer is then prompted to hold the breath at time TO. After a time period of several seconds, typically between 10 and 30 seconds, the PPG signal exhibits an increase in frequency, (decrease in IBI) 9.10, and sometimes a decrease in amplitude between AMP 3 and AMP 4. Breathing restarts between T1 and T2, with full recovery of the PPG signal at T3 when the amplitude of the PPG signal as well as IBI, 9.12, has also recovered. The time interval of variations of the PPG from normal in one or both amplitude and IBI closely equate to Tf and can therefore be used to determine the value of Tf. The sampling rate of the PPG sensor, for example, maybe at least 100 Hz with an accuracy of +/−1%.

The method chosen to determine Tf depends on which sensor or sensors are being used. If only an SpO2 sensor is available, then blood oxygen saturation (i.e. concentration) may be used. If only a PPG sensor is available, then changes in IBI and possibly pulse amplitude may be used. If both sensors are available, Tf is determined by the mean of the two values for each forced breath hold. It is usual to measure Tf n times during LG determination where n lies in the range 3-10. In this case, Tf is calculated from the mean of all n values after removing all values that lie outside the standard deviation of the set or using other appropriate statistical methods.

As mentioned above, the determination of the forced breath-hold time, Tf, is a step in assessing the LG of an individual's breathing control system. The method for calculating Tf may be contingent upon the type of sensor available during the measurement process. When utilizing an SpO2 sensor, the focus is on tracking the saturation of oxygen in the blood throughout the breath-hold exercise. Conversely, with a PPG sensor, the method revolves around observing changes in the interbeat interval (IBI) and, if applicable, alterations in pulse amplitude during the breath-hold. In scenarios where both SpO2 and PPG sensors are employed, the determination of Tf is refined by computing the average of the values obtained from each sensor for each individual forced breath-hold. This dual-sensor approach aims to enhance the accuracy of the measurement by integrating data from two distinct physiological responses. As noted above, the protocol for measuring Tf typically involves multiple iterations of the breath-hold, with the number of repetitions, denoted as ‘n’, falling within the range of 3 to 10. This repetition ensures the reliability of the data by accounting for variability in the individual's physiological responses. The mean value of Tf is then calculated from this series of measurements. To ensure statistical robustness, any outlier values that deviate beyond the standard deviation of the dataset are excluded from the calculation. Alternatively, other statistical methods may be applied to ascertain a precise and representative value of Tf, which serves as basis for the subsequent determination of LG.

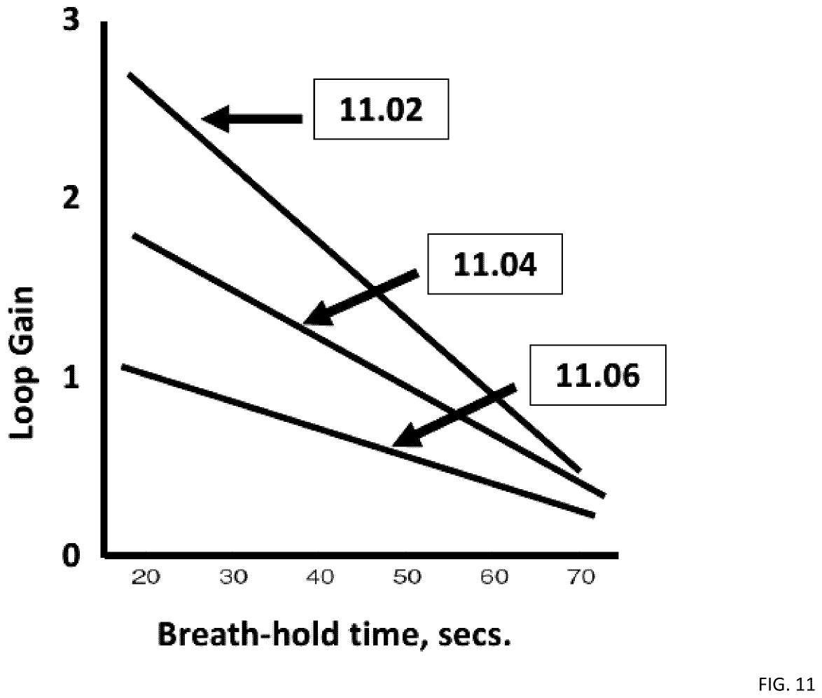

The relationship between Tf and LG is defined in a set of look-up tables determined empirically from measurements on a statistically relevant population of at least N (e.g. 10) phenotype classes. Each table in the set relates to a phenotype of one of these classes. An example of such a table is shown as a chart in FIG. 11. Phenotype matching is based on individual personal and physiological data known to be correlated with SDB including but not limited to body mass index (BMI), age, gender, exercise activity level, smoking level, asthma intensity etc. The relationship between LG and forced breath-hold time, Tf for three different phenotype categories, 11.02, 11.04, and 11.06 is shown. Once a sufferer has been assigned to a phenotype category and Tf determined by the methods disclosed herein, LG can be determined from such charts.

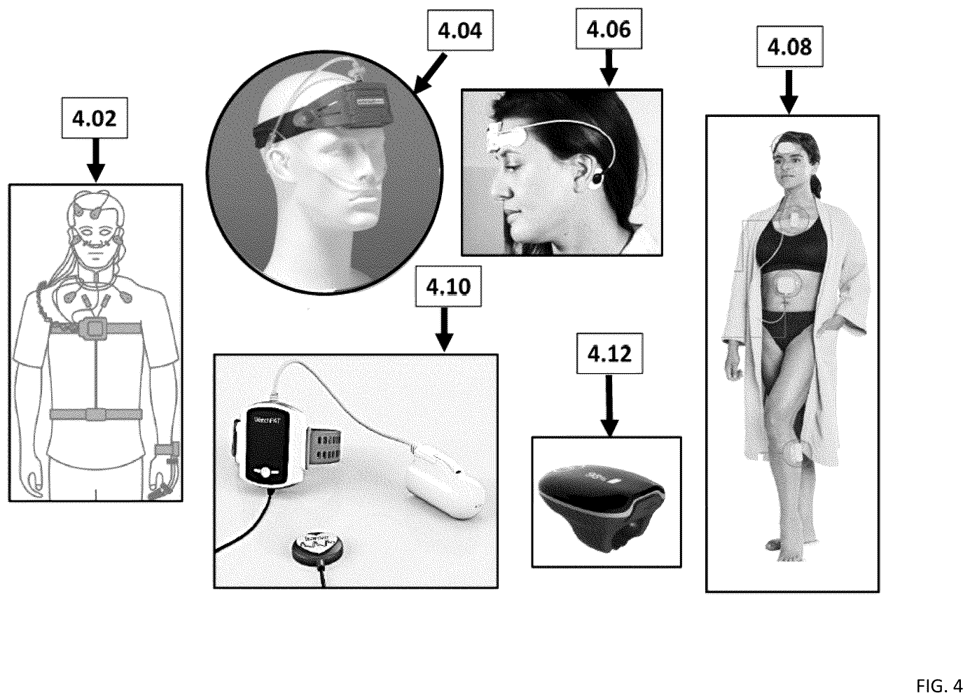

Sensors suitable for the determination of LG already exist in devices and systems for diagnosing SDB. Such systems are found in overnight sleep laboratories located away from home. Increasingly, such diagnosing systems are being deployed for home sleep testing, (HSAT's); a selection of such system configurations are shown in FIG. 4. 4.02 is a multi-component system including sensors placed at the nostrils, face, chest, wrist and fingers. A somewhat simpler system is in 4.08 with sensors placed at the face, chest, abdomen, and calf. 4.04 and 4.06 have sensors on the forehead supplemented with a nasal cannula in 4.04 and a neck sensor in 4.06. The system in 4.10 has sensors placed at the chest, wrist and finger. A less invasive configuration is the use of ring 4.12. These devices may be connected wirelessly to data processors which may include smart devices (e.g. smartphones, etc.), network servers and the like.

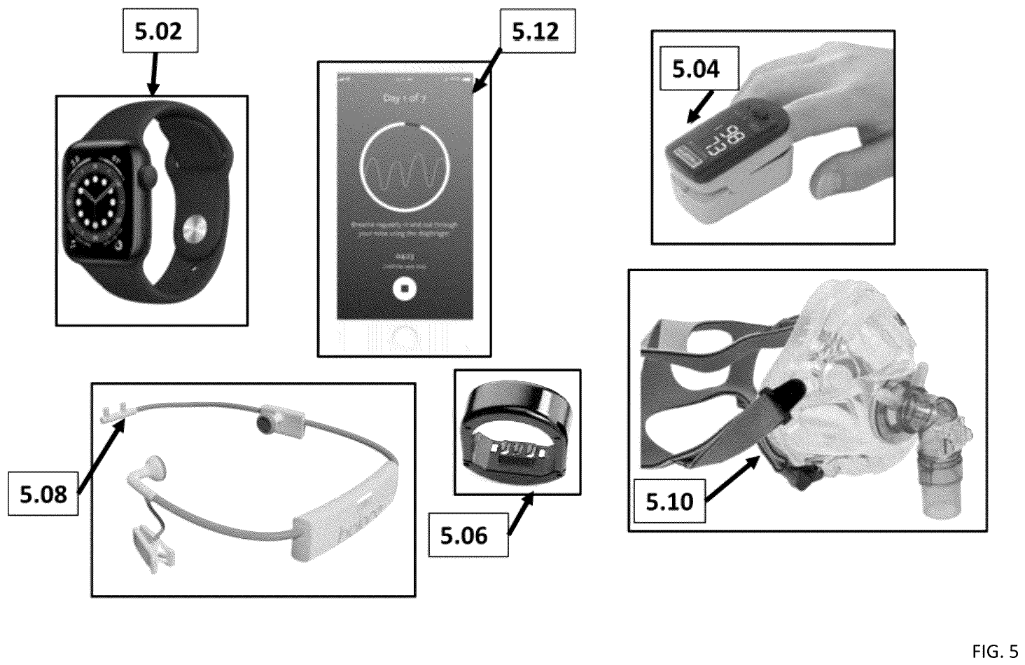

Sensors suitable for the determination of LG already exist in other portable devices not used within a full functioning HSAT for diagnosing SDB. These devices can be adapted to determine LG using the methods described herein. Examples of such devices including portable smart devices are shown in FIG. 5. 5.02 is a smart-watch heart rate sensor, 5.04 is a finger-tip pulse oximeter and heart rate sensor, 5.06 is a sensing ring for sensing heart rate, 5.08 is a headset with a nasal cannula and an car-clip sensor. 5.10 is a CPAP mask, already used for SDB therapy which, using the methods herein, can add LG diagnostics to the functionality. 5.12 is a smart phone fitted with a camera which can detect pulse data (heart rate, etc.). These devices may be connected wirelessly to data processors which may include smart devices (e.g. smartphones, etc.), network servers and the like.

In one embodiment a sufferer wears a ring 5.06, and follow the instructions delivered via a smart phone which communicates with the ring using a Bluetooth™ protocol: “Sit upright and comfortably. Breathe regularly and normally for 3 minutes. When you hear three beeps, exhale gently and hold your breath until the first clear urge at your diaphragm to breathe is felt. Inhale through the nose trying not to over breathe or gasp. Return to normal breathing until you hear three beeps after about 5 minutes. Exhale gently and hold your breath until the first clear urge to breathe is felt. Inhale through the nose trying not to gasp. Return to normal breathing until you hear three beeps in about 5 minutes. Hold the breath until the first clear urge to breathe is felt. Then take the first breath through the nose calmly. Try not to over-breathe or gasp. “(The breath-holding cycle is repeated three more times.) After the fifth breath-hold return to regular breathing. Your test is now complete.”

During this test, the ring has detected both pulse data and SpO2 data and transmitted them wirelessly to a processor such as a smart phone. The data is processed either by the local processor or by a central processor located in the cloud. The data is transmitted with appropriate encryption.

The pulse rate data is evaluated to determine five values of Tfp breath holding times derived from pulse data. The standard deviation and mean of, for example five values are calculated. Any value of Tfp outside the standard deviation from the mean is removed, and the mean value of the remaining Tfp's is calculated as Tfpfinal.

The SpO2 data is also used to determine, for example, five values of Tfs breath holding times. The standard deviation and mean of the five values are calculated. Any value of Tfs outside the standard deviation from the mean is removed, and the mean value of the remaining Tfs's is calculated as Tfsfinal. The mean of Tfpfinal and Tfsfinal is calculated to give Tffinal. LG is then determined from a look up table or chart matching the phenotype of the sufferer as in FIG. 11.

If the device is able to detect only IBI or SpO2 with the accuracy demanded, Tffinal is derived from one of the sensors parameters. In such a case, n values of Tf, where n is in the range 2-10, are measured, the standard deviation from the mean taken, those values outside the standard deviation removed and the mean of the remaining values calculates before LG is derived.

In a second embodiment a sufferer wears a CPAP mask, 5.10, and follows these example instructions delivered via a display and/or a sound source embedded the CPAP mask or in a separate device such as a smart phone. 5.12 which communicates with 5.10 using a Bluetooth™ protocol as follows: “Sit upright and comfortably. Breathe regularly and normally for 3 minutes. When you hear three beeps, exhale gently and hold your breath until the first clear urge at your diaphragm to breathe is felt. Inhale through the nose trying not to over breathe or gasp. Return to normal breathing until you hear three beeps after about 5 minutes. Exhale gently and hold your breath until the first clear urge to breathe is felt. Inhale through the nose trying not to gasp. Return to normal breathing until you hear three beeps in about 5 minutes. Hold the breath until the first clear urge to breathe is felt. Then take the first breath through the nose calmly. Try not to over-breathe or gasp. “(The breath-holding cycle is repeated three more times.) After the fifth breath-hold return to regular breathing. Your test is now complete.”

During this test, the CPAP mask detects one or more of ventilation airflow, pulse data and SpO2 data and stores the data in a within the CPAP mask, or in a local processor that communicates with the CPAP mask, or the data is transmitted to a central processor located in the cloud. The data may be transmitted with appropriate encryption protocol.

The flow rate sensor may store the maximum value of the inhale flow rate during either or both the initial and the final periods when the sufferer is undertaking relaxed breathing sequences, The initial and final sections of these sequences are discarded for between 5-15 seconds to ensure that transition periods are not included. The mean value of maximum inhale flow rate, VRrest is calculated. The maximum inhalation flowrate for the second inhalation after each breath-hold sequence is recorded, VRbh2 and the mean and standard deviation of the mean for all values during the test calculated. Those values of VRbh2 lying outside the standard deviation are discarded and the mean re-calculated to give VRbh2final. LG is then calculated as follows from a chart as shown in FIG. 10.

If LG is also determined using SpO2 and/or PPG signals, LG is calculated as the weighted mean of all values determine, as for example:

$$LG ( final ) = ( x * LG ( Flowrate ) + y * LG ( Sp O 2 ) + z * LG ( PPG ) ) / 3$$

where x, y, z are weighting factors.

These will normally be unity, but if multiple records over time indicate greater accuracy results from one method of determining LG, the factors can be adjusted appropriately.

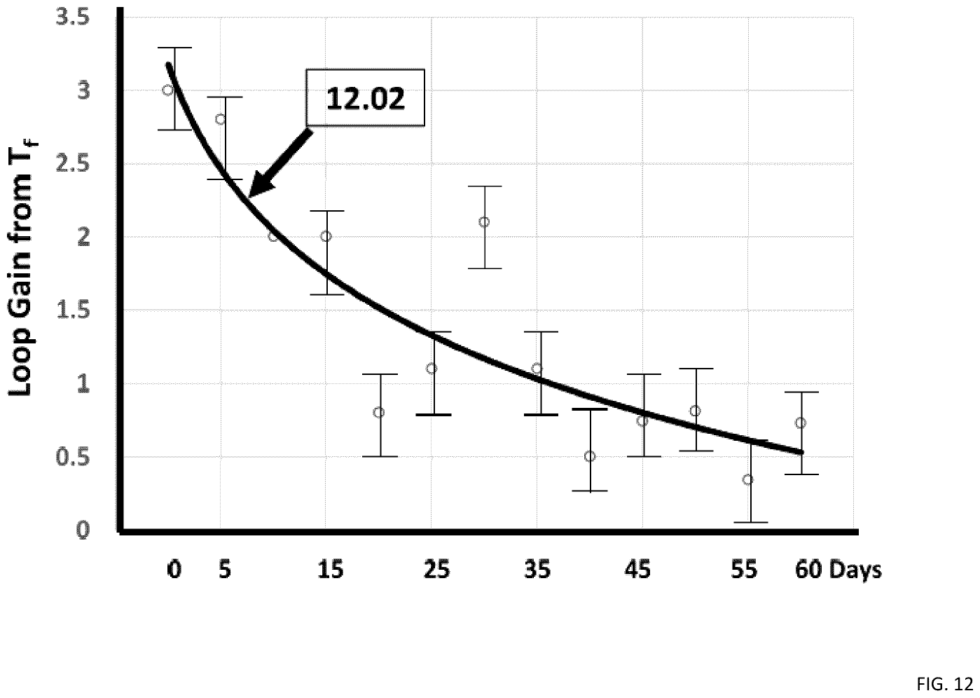

If LG is greater or equal to 0.8, it is likely that OSA symptoms may be a result partly or totally of the breathing control system being unstable from time to time. In this case, breath training as described in U.S. Pat. No. 9,830,832 may be recommended. FIG. 12 is a chart 12.02 of how LG may decline when LG is a cause of OSA. Error bars show standard deviations of each LG value. The decline in LG value over time decays logarithmically with a time constant determined by a drop (e.g., 50% drop) in LG of between 30 and 60 days.

Such breath training may be performed as a single therapeutic method or combined with other methods. When combined with CPAP therapy, the CPAP mask can be used for LG detection in the waking state using the methods disclosed here, CPAP drop detection of LG, airway patency therapy, and breath training in the waking state.

Any sensing device using the methods herein can be used to measure LG over time to track reductions of LG resulting from breath training and other therapies.

As mentioned above, LG is based on the user's ability to recover to normal breathing and physiological parameters following a perturbation, such as a voluntary breath-hold. During the breath-hold, the body's response to the lack of ventilation is monitored, and the subsequent recovery phase is analyzed. LG is a measure of the stability of the breathing control system; it reflects how well the system compensates for the disturbance and returns to a balanced state. A lower LG indicates a stable system that can effectively return to normal breathing and physiological parameters after a perturbation, while a higher LG suggests a system that overreacts, leading to instability and potential perpetuation of breathing irregularities. The recovery to normal breathing patterns and the normalization of physiological parameters like heart rate and blood oxygen saturation are thus beneficial to determining the LG value.

Determining the LG involves analyzing physiological responses to breath-holding exercises or involuntary breath-holding using various sensors. For example, heart rate sensors gauge changes in pulse rate variability and rhythm, which fluctuate in response to breath-holding, reflecting the body's compensatory mechanisms that are indicative of LG. Oximeters, specifically pulse oximeters, measure the drop in blood oxygen saturation (SpO2) during the breath-hold and monitor the recovery phase, providing a direct correlation to LG as the body's oxygen levels are a sensitive indicator of respiratory control system stability. Flow rate sensors measure the actual volume and speed of breaths, capturing the immediate impact of breath-holding on airflow, which is then used to calculate LG by comparing pre-and post-breath-hold flow rates. The integration of data from these sensors—heart rate, SpO2, and/or flow rate—enables a multifaceted assessment of the respiratory system's stability, leading to a precise determination of LG. This integrated approach ensures that the LG value reflects the complex interplay of physiological factors that govern breathing stability.

The integration of data from one or more sensors, such as heart rate sensors, blood oxygen sensors, and flow rate sensors, provides a synergistic approach to determining loop gain (LG). By combining the distinct physiological responses captured by each sensor during breath-holding exercises, a composite analysis can be performed. This analysis may involve weighted algorithms that account for the relative contributions of pulse rate variability, oxygen saturation levels, and airflow metrics. The fusion of these data streams enhances the precision of LG estimation, as it encapsulates a holistic view of the respiratory system's dynamics. Consequently, the combined sensor approach not merely corroborates the individual sensor readings but also compensates for any potential anomalies or inaccuracies, thereby yielding a more robust and reliable measure of LG.

While the foregoing is directed to example embodiments described herein, other and further example embodiments may be devised without departing from the basic scope thereof. For example, aspects of the present disclosure may be implemented in hardware or software or a combination of hardware and software (e.g. processors, memory devices, electronic circuits, and the like of the PAP device, sensors, smartphone, servers, etc.). One example embodiment described herein may be implemented as a program product for use with a computer system. The program(s) of the program product define functions of the example embodiments (including the methods described herein) and can be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed example embodiments, are example embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A portable smart device, comprising:
a sensor coupled to the device, the sensor configured to sense a physiological parameter of a user; and
a processor configured to:
perform a breath training session to determine breathing loop gain in an awake state by instructing the user to breathe in a specified manner,
receive physiological data from the sensor, the physiological data representing the sensed physiological parameter of the user, and
determine a value of the loop gain based on the physiological data from the sensor.

2. The portable smart device of claim 1, wherein the sensor is at least one of a heart rate sensor or a blood oxygen saturation sensor.

3. The portable smart device of claim 1, wherein the processor is further configured to guide the breath training session by instructing the user to breathe in a specified manner by performing breath holds and rhythmic breathing patterns intended to reduce the loop gain.

4. The portable smart device of claim 1, wherein the processor is further configured to track changes in the loop gain over time.

5. The portable smart device of claim 1, wherein the processor is further configured to determine a value of the loop gain based on a phenotype classification of the user.

6. The portable smart device of claim 1, wherein the sensor is integrated directly into the portable smart device.

7. The portable smart device of claim 1, wherein the sensor is external to and in communication with the portable smart device.

8. The portable smart device of claim 1, wherein the processor is further configured to instruct the user to perform breathing exercises for the determination of the loop gain.

9. The portable smart device of claim 1, wherein the processor is further configured to perform a classification by classifying the user as having a high loop gain based on the determined value of the loop gain and previously determined physiological data.

10. The portable smart device of claim 9, wherein the processor is further configured to tailor breath training exercises specifically to reduce the high loop gain, the tailoring based on the classification of the user and the determined value of the loop gain.

11. A method for determining breathing loop gain in an awake state using a portable smart device, the method comprising:
sensing a physiological parameter of a user with a sensor coupled to the device;
performing a breath training session to instruct the user to breathe in a specified manner;
receiving physiological data from the sensor, the physiological data representing the sensed physiological parameter of the user; and
determining a value of the loop gain based on the physiological data from the sensor.

12. The method of claim 11, wherein the sensing step involves using at least one of a heart rate sensor or a blood oxygen saturation sensor.

13. The method of claim 11, further comprising guiding the breath training session by instructing the user to breathe in a specified manner by performing breath holds and rhythmic breathing patterns intended to reduce the loop gain.

14. The method of claim 11, further comprising tracking changes in the loop gain over time.

15. The method of claim 11, further comprising determining a value of the loop gain based on a phenotype classification of the user.

16. The method of claim 11, wherein the sensor is integrated directly into the portable smart device.

17. The method of claim 11, wherein the sensor is external to and in communication with the portable smart device.

18. The method of claim 11, further comprising instructing the user to perform breathing exercises for the determination of the loop gain.

19. The method of claim 11, further comprising performing a classification by classifying the user as having a high loop gain based on the determined value of the loop gain and previously determined physiological data.

20. The method of claim 19, further comprising tailoring breath training exercises specifically to reduce the high loop gain, the tailoring based on the classification of the user and the determined value of the loop gain.
